(19) United States
(12) Patent Application Publication
(10) Pub. No.: US 2025/0120582 A1
(43) Pub. Date: Apr. 17, 2025

(54) CANCER DIAGNOSIS METHOD AND SYSTEMS USING SECOND HARMONIC GENERATION MEASUREMENTS

(71) Applicant: Arizona Board of Regents on Behalf of the University of Arizona, Tucson, AZ (US)

(72) Inventors: Jenna Montague, Tucson, AZ (US); Jennifer Barton, Tucson, AZ (US); Hasina Shir, Tucson, AZ (US)

(21) Appl. No.: 18/730,460

(22) PCT Filed: Jan. 23, 2023
(86) PCT No.: PCT/US2023/061088

§ 371 Date: Jul. 19, 2024

Related U.S. Application Data
(60) Provisional application No. 63/301,913, filed on Jan. 21, 2022.
(60) Provisional application No. 63/367,948, filed on Jul. 8, 2022.

(51) Int. Cl. A61B 1/06; A61B 1/005; A61B 1/31; A61B 5/00

(52) U.S. Cl. CPC A61B 1/0661; A61B 1/005; A61B 1/31; A61B 5/0062; A61B 5/0082; A61B 5/7264

(57) ABSTRACT

Methods, devices and systems are described that enable identification of cancerous or pre-cancerous colon tissue. The described methods and systems rely on non-imaging. randomly sampled second harmonic generation (SHG) intensity measurements that are sufficient for differentiating between tumor and normal tissue. The disclosed methods and system further provide for angle-resolved SHG measurements associated with forward and backward scattered light that eliminate the need to obtain paired normal and cancer-suspicious measurements in each patient using the described techniques.

### CROSS-REFERENCE TO RELATED APPLICATION(S)

This application claims priority to the provisional application with Ser. No. 63/301,913 titled “NON-IMAGING, RANDOM-SAMPLING SECOND HARMONIC GENERATION MEASUREMENTS TO DISTINGUISH COLON CANCER,” filed Jan. 21, 2022, and the provisional application with Ser. No. 63/367,948 titled “ANGLE-RESOLVED SECOND HARMONIC GENERATION MEASUREMENTS FOR CANCER DIAGNOSIS,” filed Jul. 8, 2022. The entire contents of the above noted provisional applications are incorporated by reference as part of the disclosure of this document.

### STATEMENT REGARDING FEDERALLY SPONSORED RESEARCH OR DEVELOPMENT

This invention was made with government support under Grant No. EB020605 awarded by the National Institutes of Health. The government has certain rights in the invention.

### TECHNICAL FIELD

The technology in this patent document relates to methods, devices and systems for identifying suspicious colon regions based on second harmonic generation light measurements.

### BACKGROUND

A global study on cancer incidence and mortality found that colorectal cancer (CRC) was the third most diagnosed cancer in the world, and it was the second leading cause of cancer death. The current standard diagnostic technique for colon cancer is a colonoscopy. Although colonoscopy can detect polyps with relatively high accuracy (93.4% sensitivity and 97.8% specificity), it fails to provide an accurate diagnosis for colorectal tumors (56.9% sensitivity and 97.8% specificity). Colonoscopes have a small working channel through which diagnostic tools can be easily implemented, which could reduce the margin for error without adding much complexity to the procedure. However, this high false-negative rate indicates a strong need for additional diagnostic techniques to verify whether a negative CRC diagnosis on a polyp is accurate.

### SUMMARY

The disclosed technology, among other features and benefits, illustrate that non-imaging, randomly sampled second harmonic generation (SHG) intensity measurements are sufficient for differentiating between tumor and normal tissue, and they further use non-imaging, random-sampling second harmonic generation measurements of a colon tissue sample to determine whether or not tumor or tumor-adjacent tissue exists in the tissue sample. The disclosed embodiments further provide for angle-resolved SHG measurements that eliminate the need to obtain paired normal and cancer-suspicious measurements in each patient.

One example endoscopic system includes an endoscope configured to be inserted into a colon of a patient to illuminate a section of the colon with incident light and receive second harmonic generation (SHG) light from the section of the colon. The endoscopic system also includes a processor capable of executing computer-readable instructions and a memory comprising computer-readable instructions for: receiving information representative of intensity values associated with the SHG light received from the section of the colon in response to illumination by an incident light; discarding information representing intensity values below a first threshold or above a second threshold to obtain a reduced data set; randomly selecting a subset of the reduced data set and comparing intensity values corresponding to the randomly selected subset to one or more predetermined values; and determining, based the comparing, whether the section of the colon includes a suspicious tissue or a normal tissue.

Another example endoscopic system includes an excitation subsystem including at least one lens positioned to receive light from a light source and to provide incident light for illuminating a section of a colon. The endoscopic system also includes an emission subsystem including collection optics configured to collect second harmonic generation (SHG) light from the section of the colon. The emission subsystem includes collection optics having multiple numerical apertures (NAs) that allow simultaneous collection of forward light representing forward scattered light and backward light comprising at least a portion of the backward scattered light from the section of the colon. The endoscopic system further includes a processor and a memory including instructions stored thereon. The instructions upon execution by the processor cause the processor to: receive information representing measurements of optical signals associated with the multiple numerical apertures in response to illumination the section of the colon with the incident light, determine a ratio associated with the forward and the backward light based on the received information, and generate an assessment regarding presence of a normal or suspicious region in the section of the colon.

### BRIEF DESCRIPTION OF THE DRAWINGS

FIG. 1 shows a schematic of the system for distinguishing colon tumor and/or cancer tissue from regular colon tissue in a patient in accordance with an example embodiment.

FIG. 2 shows a flow chart of a method for distinguishing colon cancer tissue from regular colon tissue in a patient in accordance with an example embodiment.

FIG. 3 shows a simplified diagram of the multiphoton microscopy imaging setup implemented in experimentation in accordance with an example embodiment.

FIGS. 4A-4C show representations of a colon mucosa analysis algorithm with enhanced brightness and contrast. FIG. 4A shows a raw image, FIG. 4B shows thresholded out low-and high-value pixels, and FIG. 4C shows a random assortment of those pixels. The randomly sampled image shows a sample size of 10,000 pixels for easy visualization of the algorithm. Smaller sample sizes (10, 100, and 1000) were implemented in actual data processing.

FIG. 5 shows an example graph of mean SHG pixel values in images of normal, tumor-adjacent, and tumor tissue.

FIGS. 6A-6C show example SHG images of normal (FIG. 6A), tumor-adjacent (FIG. 6B), and tumor (FIG. 6C) mucosal structures in the same subject. These images have been thresholded and have the same brightness and contrast settings to allow for a true intensity comparison.

FIG. 7 shows an example line graph of P-value versus the number of pixels sampled between Adjacent/Tumor, Normal/Adjacent, and Normal/Tumor groups. Error bars represent SEM of p values for four sets of randomly selected pixels. Significance was maintained for Normal/Adjacent and Normal/Tumor comparisons with a random sampling of >1000 pixels per image.

FIG. 8 shows example images (.jpg compression) for each subject at normal, adjacent, and tumor locations. Diagnosis is the clinical diagnosis provided to the patient. Pathology is the pathologist's description of the resected section and may not accurately reflect the samples provided to the investigators.

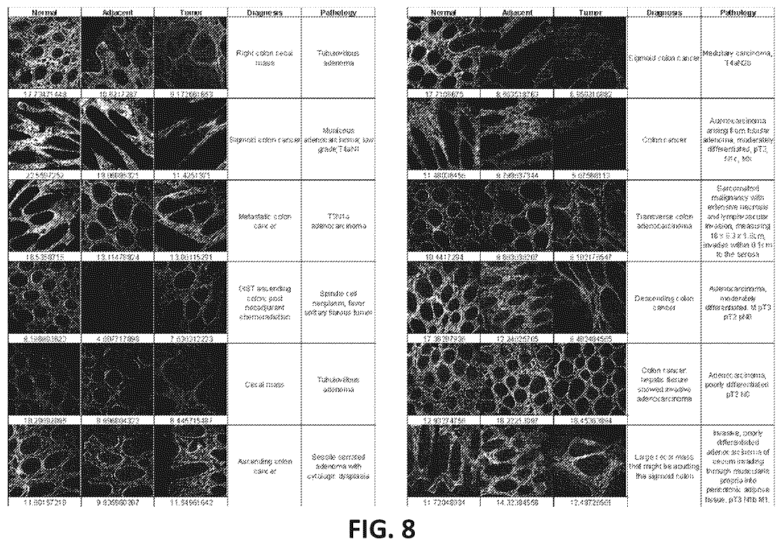

FIG. 9 illustrates the concept of forward and backward scattering associated with a sample.

FIG. 10 is a simplified diagram illustrating various interactions of light with a sample (e.g., colon) that is illuminated by the incident light.

FIG. 11 is a diagram illustrating the concept of numerical aperture using three different lenses with differing focal lengths.

FIG. 12 illustrates an optical waveguide (e.g., a dual clad optical fiber) and a multi-NA lens that can be used as the collection optics in an endoscopic system in accordance with an example embodiment.

FIG. 13 illustrates three different configurations of a multi-NA lens in accordance with example embodiments.

FIG. 14 illustrates example emission apertures with different aperture sizes in accordance with some example embodiments.

FIG. 15 illustrates one example pattern of the measured intensity versus NA for different collagen gels emulating normal tissue and non-normal tissues.

FIG. 16 illustrates a set of operations that can be carried out to determine a presence of a suspicious tissue in a colon in accordance with an example embodiment.

FIG. 17 illustrates a set of operations for identifying a suspicious or a normal tissue in a colon in accordance with an example embodiment.

### DETAILED DESCRIPTION

Tumor cells grow in an uncontrolled manner, because of a biochemical change in the nutrient supply from their microenvironment, the extracellular matrix. Collagen, a major structural component in the extracellular matrix, has been shown to have a pivotal role in cancer development. Studies have shown that structural changes happen in the collagen surrounding a tumor that can be difficult to detect with traditional histology. Additional methods for studying the structural changes in collagen associated with cancer development can provide insight into cancer diagnosis and progression status.

Second harmonic generation (SHG) is used to quantitatively study collagen's structural changes. SHG is a multiphoton optical phenomenon that certain nonlinear materials, including collagen, can produce through a scattering mechanism. When two photons of the same frequency simultaneously interact within these materials, one photon with double the energy and half the wavelength of the original photons may be produced. The intensity of the SHG signal that collagen can produce is dependent on the nonlinear optical susceptibility of the collagen. Nonlinear optical susceptibility depends on the thickness of fiber thickness, fiber density, and fibril “packing” which is the three-dimensional structure of the fibrils that make up collagen fibers. Many studies have used backscattered SHG as a quantitative indicator of extracellular matrix alterations in various cancer types, including CRC. Researchers have also shown that an SHG directionality comparison between the forward-and backward-scattered signal can be utilized ex vivo as an independent prognostic indicator for metastasis.

There are two main advantages to studying collagen with SHG. The primary advantage is that it can serve as a label-free imaging technique that can optically isolate collagen from its surroundings since collagen is the dominant material in tissue that produces a significant SHG signal. Secondarily, to produce a strong SHG effect, it is necessary to confine the excitation light to a small focal volume to increase the probability of a two-photon effect taking place. Given this selective excitation only within this small focal volume, three-dimensional information about the collagen structure can be easily obtained by gathering intensity data from each focal volume and combining these individual voxels (volumetric pixels) to construct a three-dimensional image.

The data for an SHG image is traditionally obtained ex vivo with a multiphoton microscope. The key components of a multiphoton microscope are a femtosecond laser, a high numerical aperture (NA) focusing lens or objective, and scanning components that move the laser beam, and thus the focal volume, across the field of view. Scanning usually is accomplished with galvanometer-based mirrors that rotate to move the beam across the field of view. Alternative scanning methods may maximize acquisition speed such as resonant galvanometer systems, hexagonal mirror scanning, and MEMS mirrors.

Some systems have previously obtained multiphoton images and/or second harmonic generation images with an endoscope. Since scanning mirrors and MEMS devices are challenging to fit in the distal assembly of very small diameter endoscopes, a tube piezoelectric device with a cantilevered fiber was used (although other more complex scanning mechanisms can also be utilized). However, miniature scanning mechanisms are generally expensive, complex, and may be inaccurate. There is therefore a need for a simpler way to obtain SHG intensity data endoscopically. The experiments and analyses disclosed herein inter alia investigate whether scanning (e.g., electronic or synchronous scanning) is necessary to obtain meaningful SHG intensity data and illustrates that randomly sampled SHG intensity measurements are sufficient for differentiating between tumor and normal tissue in accordance with some example embodiments.

It is an objective of the disclosed technology to provide systems and methods that allow for the use of non-imaging, random-sampling second harmonic generation measurements of a colon tissue sample to determine whether or not tumor or tumor-adjacent tissue exists in the tissue sample and/or to determine whether a tumor (or neoplasm, or polyp) is benign or malignant. Embodiments of the present invention can be freely combined with each other if they are not mutually exclusive.

Globally, colorectal cancer was the second leading cause of cancer death in 2020. Research suggests that collagen, a major structural protein, plays a pivotal role in cancer development and metastasis, and by extension, subject prognosis. Collagen surrounding tumor cells undergoes structural changes that can be quantitatively studied with second harmonic generation (SHG), a subset of multiphoton microscopy (MPM). MPM as an imaging modality is difficult to implement in an endoscope because of the complex and expensive miniaturized scanning components required. Endoscope complexity can be greatly reduced by implementing a simpler, non-synchronized scanning mechanism.

The disclosed embodiments relate, in some aspects, to systems and methods for distinguishing colon cancer tissue from regular colon tissue in a patient. In some embodiments, the system may comprise an endoscope capable of generating one or more SHG measurements of the colon. The system may further comprise a communication component operatively connecting the endoscope to a computing device. The system may further comprise the computing device capable of accepting the one or more SHG measurements from the endoscope as the endoscope is moved relative to the tissue (such as by, either manually moving the endoscope by the operator of the endoscope, or due to normal patient motion), measuring an intensity value for each SHG measurement of the one or more SHG measurements, and determining, based on the plurality of intensity values, whether or not a tumor may be present in the colon of the patient. In some cases, movement of the endoscope may be effectuated using mechanical and/or electronic devices.

In some embodiments, a method for distinguishing colon cancer tissue from regular colon tissue may comprise providing an endoscope capable of generating an SHG measurement of the colon. The method may further comprise providing a computing device operatively connected to the endoscope by a communication component. The method may further comprise directing the endoscope through a rectum of the patient into the colon of the patient, generating one or more SHG measurements of the colon by the endoscope, transmitting the one or more SHG measurements to the computing device from the endoscope, measuring an intensity value for each SHG measurement of the one or more SHG measurements through use of the computing device, and determining, based on the plurality of intensity values, whether or not a tumor may be present in the colon of the patient through use of the computing device.

One of the unique and inventive technical features of the disclosed technology is the use of non-imaging second harmonic generation measurements to distinguish tumor tissue from normal tissue. Without wishing to limit the invention to any theory or mechanism, the technical features of the disclosed embodiments advantageously provide for accurate and efficient identification of a tumor and/or malignancy in a colon region of a patient while obviating the complexity and expense of miniaturized synchronous scanning mechanisms. The disclosed technology in fact reduces the complexity and expense of systems with any scanning mechanism, miniaturized or otherwise.

Furthermore, the inventive technical features of the disclosed technology contributed to a surprising result. For example, one skilled in the art would view SHG as an imaging technique and would not expect the use of SHG as a non-imaging technique. Furthermore, one skilled in the art would expect that the random sampling algorithm or technique implemented in the disclosed embodiments would be inaccurate in comparison to fully imaging and analyzing the colon tissue region of a patient, and thus the latter method would be preferable due to the consequences of a false-negative diagnosis. Surprisingly, according to some embodiment, the SHG is implemented as a non-imaging technique that results in a similar or a lower rate of false negatives than prior imaging and scanning systems for identifying tumors in colon tissue using probabilistic sampling. Thus, the inventive technical features of the disclosed embodiments contributed to a surprising result.

Any feature or combination of features described herein are included within the scope of the present invention provided that the features included in any such combination are not mutually inconsistent as will be apparent from the context, this specification, and the knowledge of one of ordinary skill in the art.

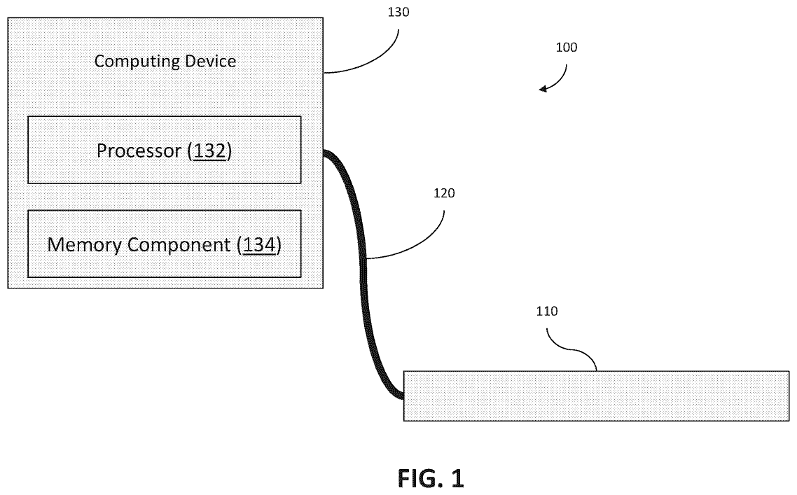

Referring now to FIG. 1, a system (100) for distinguishing colon cancer tissue from regular colon tissue in a patient is illustrated. In some embodiments, the system (100) may comprise an endoscope (110) configured to be inserted through a rectum of the patient into a colon of the patient. The endoscope (110) may be capable of generating one or more second harmonic generation (SHG) measurements of the colon. The system (100) may further comprise a communication component (120) operatively connecting the endoscope (110) to a computing device (130). The system (100) may further comprise the computing device (130) comprising a processor (132) capable of executing computer-readable instructions and a memory component (134) comprising computer-readable instructions. The computer-readable instructions may comprise instructions for accepting the SHG measurements from the endoscope (110), measuring an intensity value for each SHG measurement of the one or more SHG measurements, and determining, based on the plurality of intensity values, whether or not a tumor may be present in the colon of the patient.

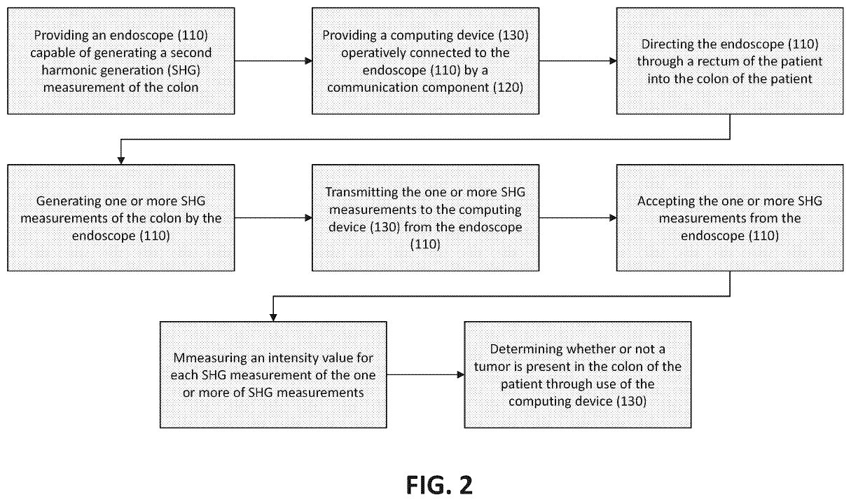

Referring now to FIG. 2, a method for distinguishing colon cancer tissue from regular colon tissue in a patient is described. In some embodiments, the method may comprise providing an endoscope (110) configured to be inserted through a rectum of the patient into a colon of the patient. The endoscope (110) may be capable of generating one or more second harmonic generation (SHG) measurements of the colon. The method may further comprise providing a computing device (130) operatively connected to the endoscope (110) by a communication component (120). The computing device (130) may comprise a processor (132) capable of executing computer-readable instructions and a memory component (134) comprising computer-readable instructions. The method may further comprise directing the endoscope (110) through a rectum of the patient into the colon of the patient. In some embodiments, the endoscope may be inserted into the rectum of the patient through a colonoscope channel to minimize invasiveness. The method may further comprise generating one or more SHG measurements of the colon by the endoscope (110), transmitting the one or more SHG measurements to the computing device (130) from the endoscope (110), measuring an intensity value for each SHG measurement of the one or more SHG measurements through use of the computing device (130), and determining, based on the intensity values, whether or not a tumor may be present in the colon of the patient through use of the computing device (130).

In some embodiments, the plurality of measurements may comprise 1000 or more measurements. Determining, based on the intensity values, whether or not a tumor and/or malignancy may be present in the colon of the patient may comprise comparing each intensity value to a threshold intensity value, calculating a number of intensity values below the threshold intensity value, and determining, based on the number of intensity values below the threshold intensity value, whether or not the tumor may be present in the colon of the patient. This may additionally allow for the categorization of normal tissue, tumor-adjacent tissue, and tumor tissue. The threshold intensity value may be about 12 on a 0 to 255 scale. The communication component (120) may comprise a wire connecting the endoscope (110) to the computing device (130). In other embodiments, the communication component (120) may comprise a wireless component disposed in the endoscope (110) capable of transmitting measurements gathered by the endoscope (110) to the computing device (130).

The following is a non-limiting example embodiment.

This study investigates whether non-imaging, randomly sampled SHG intensity measurements are sufficient to distinguish normal tissue from tumor/tumor-adjacent tissue. Unstained tumor, normal, and adjacent formalin-fixed, paraffin-embedded thin sections from 12 colorectal cancer subjects were imaged using a multiphoton microscope with 850 nm excitation and 400-430 nm emission band, constant power, and consisting of 1024×1024 pixels over 425×425μm. SHG signal from collagen fibers was isolated by grayscale thresholding, and the grayscale mean of the thresholded image was calculated. Then, random supra-threshold pixels in the image were selected. The mean SHG signal from normal samples was significantly greater than adjacent samples (p=0.014) and cancer samples (p=0.007). For both tumor and adjacent comparisons to normal tissue, the p-value becomes reliable after randomly sampling only 1000 pixels. This study suggests that reliable diagnostic information may be obtained through simple non-imaging, random-sampling SHG intensity measurements. A simple endoscope with this capability can be constructed to help identify suspicious masses or optimum surgical margins.

Surgically resected colon tissue samples from 12 colon cancer subjects were fixed in 10% formalin, embedded in paraffin, and cut into 6 μm sections using a microtome. The only reagents used were serial dilutions of ethanol for deparaffinization and water for storage. Three different samples were obtained from each subject's resected tissue: tumor, tumor-adjacent, and normal. Tumor samples were obtained from the bulk of the tumor. Tumor-adjacent samples were taken from the first normal-appearing (to the physician) tissue adjacent to the tumor. Finally, normal tissue was obtained from the edge of the resection where the physician assumed a clear margin, typically 1 cm or more away from the tumor.

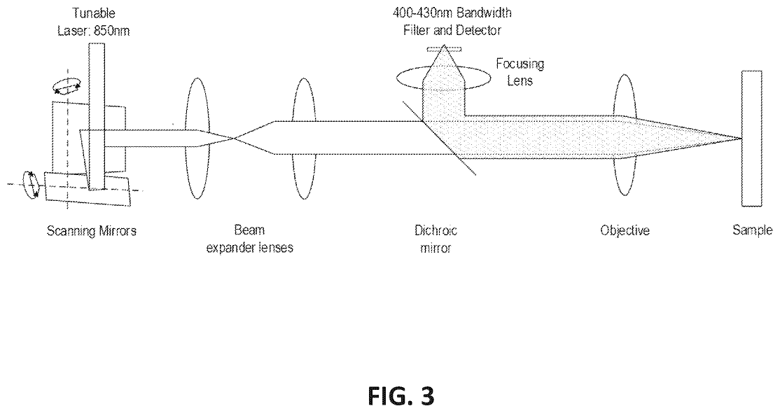

Images were obtained with a Zeiss LSM 880 multiphoton microscope with 850 nm excitation and a 400-430 nm emission band (FIG. 3). In FIG. 3, the scanning mirrors are used to scan the input beam (Tunable Laser: 850 nm in the example shown). The beam is enlarged by the beam expander lenses, is transmitted through the dichroic mirror and delivered to the sample by the objective lens. The light from the object, which is at a different wavelength (e.g., one-half the wavelength compared to the illumination light), is collected by the objective, is reflected by the dichroic mirror in the direction of a focusing lens and is received onto a detector that can include a spectral filter to capture the light at the emitted wavelength range of interest. In the above example, the objective was a 30×/0.8 NA Plan-Apochromat, and the laser was a tunable Spectra-Physics Mai Tai DeepSee laser (tunable range 690-1040 nm). The image size was 1024×1024 pixels over 425×425 μm. Power was kept constant at 1.84±0.007 W. Image planes were located manually by the user. Since slides were only 6 μm thick and axial resolution is limited to about 1-2 μm, only one image plane was obtained, resulting in two-dimensional data.

All images contained mucosal structures. The mucosa is the outermost layer of the colon epithelium, and thus the most easily accessible with an endoscope in vivo. Subjects with slides that did not contain mucosa were excluded from the study and are not included in the count of 12 subjects analyzed. One region per slide was selected by the microscope user with the only criteria being (1) image contains mucosa only and (2) no image artifacts from sectioning. Thus, regions within each slide contained a somewhat random selection of mucosa.

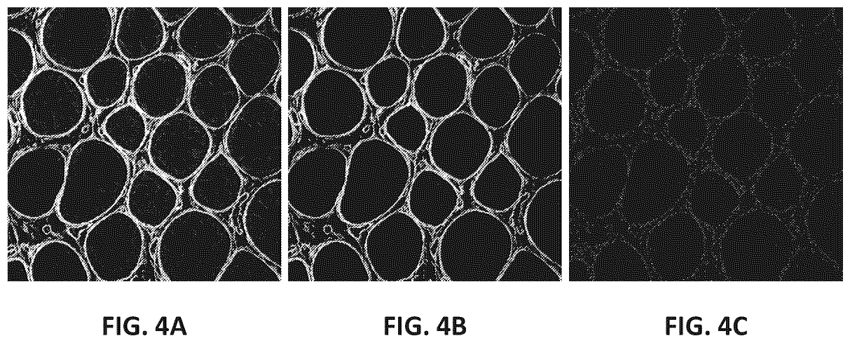

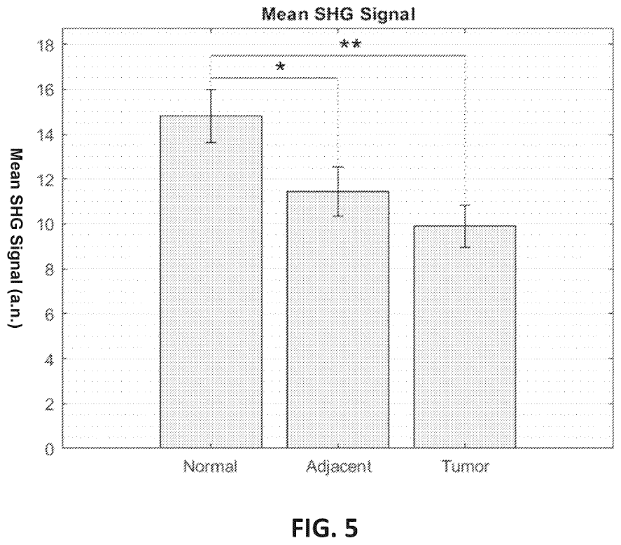

Once images were obtained, the data from each image was processed in MATLAB. As shown in FIGS. 4A-4C, a threshold was applied that assigned low pixel values (approximate intensity of autofluorescence) and greater than 254 (saturated pixels) as empty sets. The autofluorescence threshold was determined semi-subjectively; the user-determined a unique threshold for each image by determining average pixel values in a section of the background. Then, the overall average threshold (<4) was applied to every image. However, the user-determined threshold was relatively consistent across the board, with a pixel-value mean of 3.82+0.25 (SEM), and a minimum and maximum of 1 and 6. After applying the thresholds, the mean SHG intensity of each image was calculated, as illustrated in FIG. 5 using a paired t-test, p-value<0.05=*, p-value<0.01=**.

Next, to simulate a non-scanning clinical situation, a random sampling algorithm was applied. Random sampling is meant to simulate a physician's movement of the endoscope in a suspicious area that they previously identified with reflectance imaging. For the lowest sampling size, 10 pixels, an array of 10 random numbers with values between 1 and the number of thresholded pixels in the image was created with the “randperm” function in MATLAB. This array contained the indices of the selected pixels for analysis. The mean SHG intensity of the randomly selected pixels in each image was then calculated. This process was repeated at logarithmically increasing sample sizes, including 10, 100, and 1000 pixels. Larger sample sizes were not possible in this example experiment, as the thresholding step sometimes excluded many low-value pixels in the image. Lastly, images were processed through the random sampling algorithm four times to estimate variation in sampling.

Significance was found between the intensity obtained from normal and tumor tissue, and normal and tumor-adjacent tissue. The normal tissue had high intensity, while the tumor and tumor-adjacent tissue gave off less signal (with the tumor being the lowest). Qualitatively, the SHG images reflect this trend, with a lower apparent intensity of collagen fibers in tumor and tumor-adjacent images, with tumor images again having the lowest apparent intensity. Finally, when randomly-sampled, non-imaging SHG measurements were digitally simulated, significance was found with a sample size as low as 1000 pixels, suggesting that imaging is not necessary for obtaining meaningful diagnostic information.

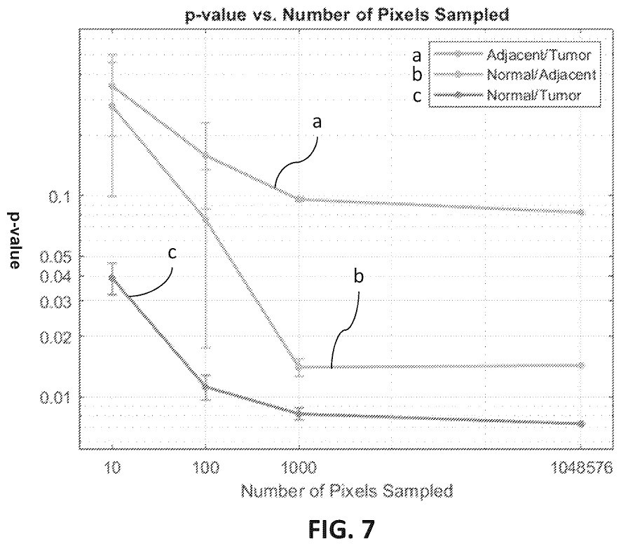

FIG. 7 shows that the overall mean SHG signal from the normal samples was significantly different from the adjacent samples (p=0.014) and the cancer samples (p=0.007), using a paired t-test. This suggests that the SHG intensity data is different in mucosal collagen images obtained in normal tissue vs. tumor and tumor-adjacent tissue. Normal tissue had the highest mean SHG intensity, followed by tumor-adjacent and tumor tissue. Although it is not statistically significant with this small data set, tumor-adjacent tissue had a higher mean SHG signal than tumor tissue.

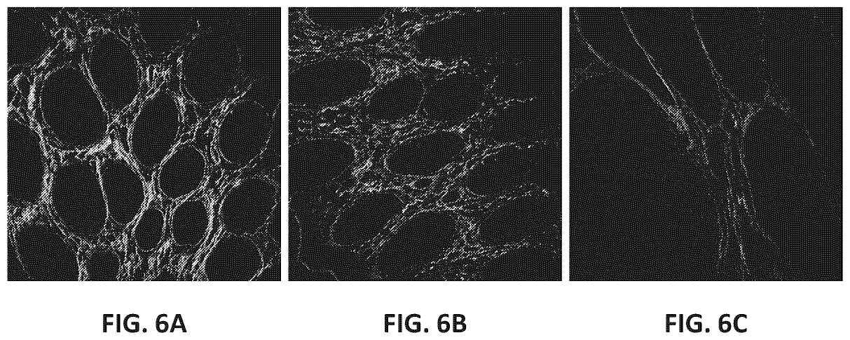

FIGS. 6A-6C show an example of normal, tumor-adjacent, and tumor tissue from a subject that represents the overall downward trend of the quantitative data previously shown in FIG. 5. Note that, in addition to the lower-density structures in the tumor, there is also a lower overall intensity of signal going from normal tissue to tumor tissue. This supports the trend shown in FIG. 5.

Upon analysis of the randomly sampled images, it was found that the p-value becomes reliable after sampling only 1000 pixels, for both the Normal/Tumor comparison and the Normal/Adjacent comparison (FIG. 7). This data supports that non-imaging, randomly-sampled SHG intensity measurements are likely to be sufficient for differentiating between normal and tumor/tumor-adjacent tissue in the colon.

The results suggest that diagnostic information about colon cancer can be found through randomly sampled simple intensity measurements of the collagen, in histological sections. These results suggest that collagen's structural changes in and around a tumor directly affect the thickness and dispersion of collagen fibers, and therefore the intensity of second harmonic intensity that is detected. The results imply that scanning to form a second harmonic image of collagen is not necessary for diagnosing colorectal cancer and therefore points to the potential development of a simplified non-imaging system.

In the above analyses, the “adjacent” slides were taken from a region that a pathologist decided had a normal appearance, yet the SHG intensity in this region was significantly lower than normal. Thus, this second harmonic measurement method supports the presence of a field-effect that influences structural changes in the collagen surrounding a tumor, which may not be apparent to gross examination.

A prior comprehensive study showed with osteosarcoma, melanoma, and breast cancer that SHG intensity is lower in tumors than in normal tissue, which is in agreement with this study on colon cancer. Limitations of this comparison include a difference in cancer type and a focus on possible endoscopic applications, and therefore mucosal/luminal regions of interest.

A different study found the opposite trend in SHG intensity in colorectal tumors. That study found that SHG intensity in tumors is higher than in normal tissue, rather than the observations described in this patent document that found that the SHG intensity in colorectal tumors is lower. Several factors could explain this variation. The most likely explanation is that the observations in this patent document focused on imaging colonic mucosa, while the other study imaged different colonic structures. Other explanations could include variations in cancer subtype, imaging system parameters (NA, laser wavelength, pulse width), or subject factors such as age or sex.

With such a high false-negative diagnostic rate for colorectal tumors, colonoscopy technology needs improvement to verify negative diagnoses. Second-harmonic generation measurements, while widely known to be effective in comparing cancerous tissue to normal tissue, have been limited primarily to ex vivo applications in part due to the complexity and expense of miniaturized scanning mechanisms. The disclosed technology with focus on colon mucosa supports that scanning to obtain an image may is not necessary to obtain enough data on a mass to predict whether it is a tumor. Second harmonic intensity alone, averaged over random pixels in an image, contains enough information to differentiate between normal tissue and tumor/tumor-adjacent tissue. The disclosed embodiments provide for implementation of SHG technology in a simple small endoscope that can be introduced through the working channel of a colonoscope to provide additional diagnostic information.

Although there has been shown and described example embodiments of the present invention, it will be readily apparent to those skilled in the art that modifications may be made thereto within the scope of the disclosed technology.

An example system (100) for distinguishing tumor and/or malignant colon cancer tissue from regular colon tissue in a patient, the system (100) includes an endoscope (110) configured to be inserted through a rectum of the patient into a colon of the patient, wherein the endoscope (110) is capable of generating one or more second harmonic generation (SHG) measurements of the colon; a communication component (120) operatively connecting the endoscope (110) to a computing device (130); and the computing device (130) comprising a processor (132) capable of executing computer-readable instructions and a memory component (134) comprising computer-readable instructions for: accepting the one or more SHG measurements from the endoscope (110); measuring an intensity value for each SHG measurement of the plurality of SHG measurements; and determining, based on the intensity values, whether or not a tumor and/or malignancy is present in the colon of the patient.

In one example, the plurality of pixels comprises 1000 or more pixels. In another example, determining, based on the plurality of intensity values, whether or not a tumor is present in the colon of the patient comprises: comparing each intensity value to a threshold intensity value; calculating a number of intensity values below the threshold intensity value; and determining, based on the number of intensity values below the threshold intensity value, whether or not the tumor is present in the colon of the patient.

In one example, the threshold intensity value is about 12 on a 0 to 255 scale of intensity. In another example, the communication component (120) comprises a wire connecting the endoscope (110) to the computing device (130). In yet another example, the communication component (120) comprises a wireless component disposed in the endoscope (110) capable of transmitting measurements gathered by the endoscope (110) to the computing device (130).

An example method for distinguishing colon cancer tissue from regular colon tissue in a patient includes: providing an endoscope (110) configured to be inserted through a rectum of the patient into a colon of the patient, wherein the endoscope (110) is capable of generating a second harmonic generation (SHG) measurement of the colon;

providing a computing device (130) operatively connected to the endoscope (110) by a communication component (120), the computing device (130) comprising a processor (132) capable of executing computer-readable instructions and a memory component (134) comprising computer-readable instructions; directing the endoscope (110) through a rectum of the patient into the colon of the patient; generating one or more SHG measurements of the colon by the endoscope (110); transmitting the one or more SHG measurements to the computing device (130) from the endoscope (110); accepting the one or more SHG measurements from the endoscope (110); measuring an intensity value for each SHG measurement of the one or more of SHG measurements; and determining, based on the plurality of intensity values, whether or not a tumor is present in the colon of the patient.

In one example, the plurality of pixels comprises 1000 or more pixels. In another example, determining, based on the plurality of intensity values, whether or not a tumor is present in the colon of the patient comprises: comparing each intensity value to a threshold intensity value; calculating a number of intensity values below the threshold intensity value; and determining, based on the number of intensity values below the threshold intensity value, whether or not the tumor is present in the colon of the patient.

In one example, the threshold intensity value is about 12 on a 0 to 255 scale of intensity. In another example, the communication component (120) comprises a wire connecting the endoscope (110) to the computing device (130). In yet another example, the communication component (120) comprises a wireless component disposed in the endoscope (110) capable of transmitting measurements gathered by the endoscope (110) to the computing device (130).

As noted earlier, for both tumor and adjacent comparisons to normal tissue, p value becomes reliable after randomly sampling only 1000 pixels (see, for example, FIG. 7 plots). This unexpected result enables identification and diagnosis of tumor and/or cancerous and/or pre-cancerous (generally “suspicious”) tissue using only the intensity (or equivalent signal) measurements at a subset of points (or pixels) rather than localizing and/or analyzing all of the measured data points. As a result, the time and computational resources are significantly reduced. In some embodiments, a random sample of only about 1% of the pre-threshold measured data points suffices to produce an assessment of the suspicious tissue with high confidence. In some embodiments, prior to performing the random sampling, data points with intensity values that are below a pretrained threshold are excluded.

The benefits of the disclosed embodiments can further be appreciated by noting that the disclosed techniques also eliminate or reduce the reliance on an observer (e.g., a lab technician, a doctor or another specialist) to make subjective assessments based on images in which identification of suspicious regions can be difficult or impossible. For example, as explained earlier, the disclosed embodiments enable the identification of adjacent regions that include tumor or pre-tumor tissue that are not visible or detectable in images that are obtained by qualitative imaging techniques. These and other advantages can be obtained using the disclosed endoscopic systems and methods that are implemented using a simple endoscope, without requiring additional optical, electrical and/or mechanical scanning components that are the hallmark of existing endoscopic imaging systems. The disclosed embodiments further facilitate the operation of the endoscope and associated systems, as well as interpretation of the results, with minimal training, since the information is objective and quantitative.

The experiments described earlier in this patent document rely on measurements and comparison of two regions. For example, the paired t test compares two measurements taken from the same individual or object that represent two different conditions (e.g., normal tissue versus suspicious tissue). While the normal tissue characteristics may be obtained from a database compiled from prior measurements of the general population (assuming that the database includes enough samples to provide meaningful information), or alternatively obtained by making a separate measurement of the normal tissue of the patient, it is beneficial to implement a capability that eliminates the need for a separate measurement of the normal (or reference) signal. In addition to improving the speed of the measurements, doing so eliminates inaccuracies that can stem from inadvertent movements of the patient and/or endoscope from one measurement to the next. Furthermore, it eliminates the reliance on a presumptive “normal” tissue characteristic based on the general population that may be unreliable and vary based on age, ethnicity, sex of the patient.

According to some embodiments, simultaneous measurements of the two regions is carried out by incorporating angle-resolved measurements that can eliminate the need to obtain paired normal and cancer-suspicious measurements in each patient. Some ex vivo thin section studies have shown that the ratio of SHG signal scattered in the forward (transmission) vs. backward (reflection) direction, or F/B ratio, can predict cancer metastasis. In semi-infinite tissue encountered in vivo, photons which initially scatter forward likely undergo more scattering events before being remitted from the tissue, thus emerging on average at a greater angular subtense. As explained in further detail below, the improved techniques disclosed in some embodiments herein rely on differing ratios of smaller vs. larger remittance angle for normal and suspicious colon tissues to facilitate identification of suspicious regions based on simultaneous measurements of normal and suspicious regions.

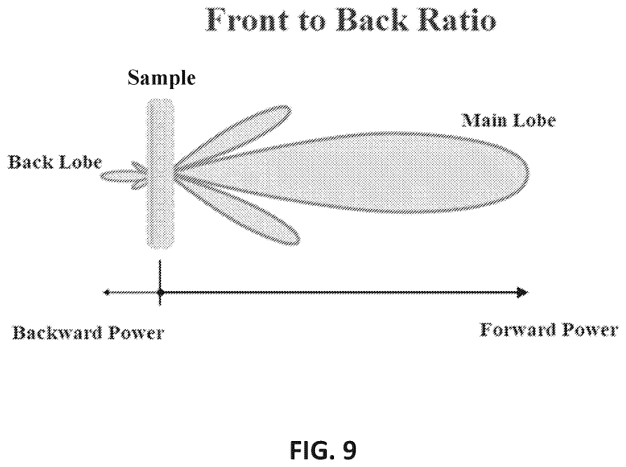

To facilitate the understanding of the disclosed embodiments, FIG. 9 illustrates the concept of forward and backward scattering associated with a sample. In this example, by measuring the amount of light that scatters forward (F) and the light that scatters backward (B), the F/B ratio can be computed and used to identify normal vs. suspicious regions. This simple example, however, cannot be used for identifying suspicious regions of the colon via endoscopic systems for the simple reason that the measurement of the forward scattered light transmitted through the opposite side of the colon wall is not practical unless the patient's colon is exposed (e.g., during surgery).

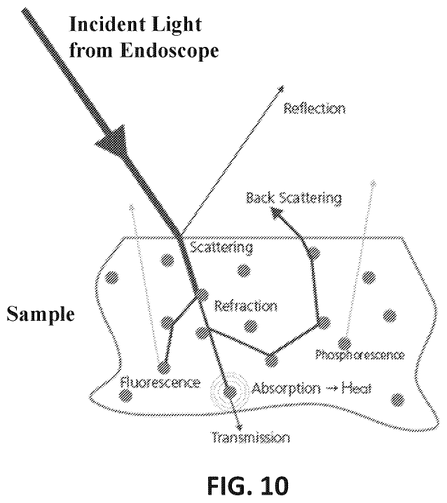

FIG. 10 is a simplified diagram illustrating various interactions of light with a sample (e.g., colon) that is illuminated by the incident light. A portion of the incident light is reflected from the surface of the sample. Another portion of the incident light penetrates the sample and can interact with one or more scattering sites, causing at least a portion of the light to be (a) transmitted through the sample at the back side, (b) emerge from the sample through the front side (i.e., same side that is illuminated), and/or (c) get absorbed (e.g., converted to heat) within the sample. As illustrated, part of the light that penetrates the sample can also be reemitted via fluorescence and/or photo phosphorescence. It can be generally observed that photons tend to initially scatter forward and undergo multiple scattering events before emerging from the front side. The multiple scattering events cause the scattered light to preferentially emerge within a wider collection angle, while the truly backward scattered light emerges at a narrower angle.

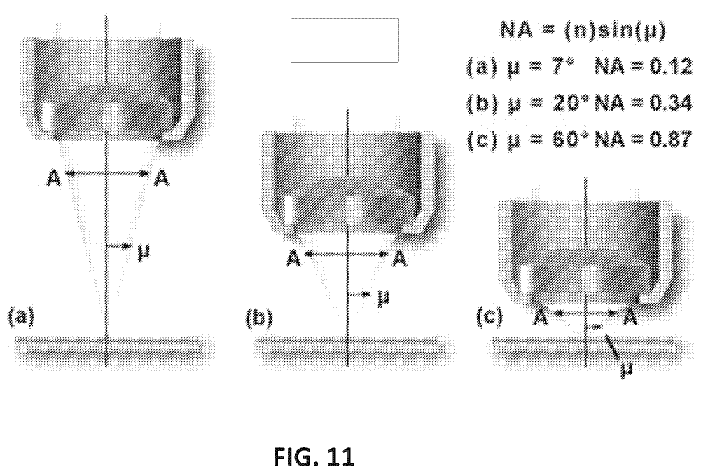

The disclosed technology can be implemented in various embodiments to use the backscattered light that emerges from the front side of the sample as both the “backward” light and the “forward” light in obtaining the F/B ratio. In particular, backscattered light, when measured with small collection angles, represents the backward light, and when measured using larger collection angles represents the forward light. The latter follows from the general principle that photons associated with the forward scattered light initially scatter forward and undergo multiple scattering events before emerging from the front side. The multiple scattering events cause the light to emerge at wider angles. Therefore, a single measurement of the backscattered light using collection optics with two or more different collection angles or numerical apertures (NAs) enables the F/B ratio to be determined, as further explained below. FIG. 11 is a diagram illustrating the concept of numerical aperture using three different lenses with differing focal lengths. As evident from FIG. 11, a larger cone of light can be collected by switching to a lens with a higher numerical aperture.

In this patent document, the term forward light is used to convey a portion of the backscattered light that represents (or is a proxy of) light scattered in the forward direction. The term backward light is used to convey a portion of the backscattered light that represents light scattered in the backward direction. The forward and backward light are typically used to determine the F/B ratio. According to some embodiments, collection optics in an endoscopic system with multiple collection-angle capabilities are implemented to simultaneously measure the forward and backward light from a sample. In particular, a larger collection angle allows the measurement of the forward light and a smaller collection angle enabled the measurement of the backward light. Additionally, forward light that is multiply scattered and then emerges from the front face of the tissue not only has, on average, a higher remitted angle, it is also remitted at a larger distance from the incident beam. Accordingly, the differences in remitted distance can be used additionally, or alternatively, to the collection angle criteria, to distinguish the forward vs. the backward scattered SHG light.

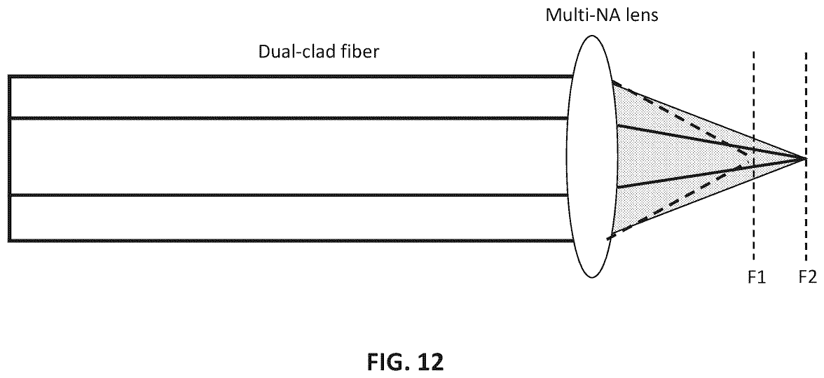

FIG. 12 illustrates an optical waveguide (e.g., a dual clad optical fiber) and a multi-NA lens that can be used as the collection optics in an endoscopic system in accordance with an example embodiment. Different NAs can be achieved by having the same beam diameter focused to two different focal lengths, or by having a smaller beam diameter focused to the same focal length. FIG. 12 illustrates both options. The dashed line and by the shaded region represent the scenario where the same beam diameter is focused at different focal distances. The solid line and the shaded region depict the scenario where different beam diameters are focused to same focal distance (or light from the same lateral position is collected at different angles). The latter is particularly useful since the light produced by the object (e.g., a section of the colon) emanates from one focal distance. Using this configuration, the higher NA section(s) of the multi-NA lens captures the forward and the lower NA section(s) of the multi-NA lens captures the backward light. In fact, in the dual (or triple in some configurations) clad fiber configuration, the cladding(s) collect light at both larger angles and larger distances than the core. In some configurations, both the forward light that is captured via a larger collection angle and the backward light that is captured via a smaller collection angle can include other types of emergent light, such as all or some of the backward light (in forward light measurements), reflected light, fluorescence and/or phosphorescence light. In some embodiments, some or all of these additional light components can be removed or blocked optically and/or electronically prior to determining the F/B ratio.

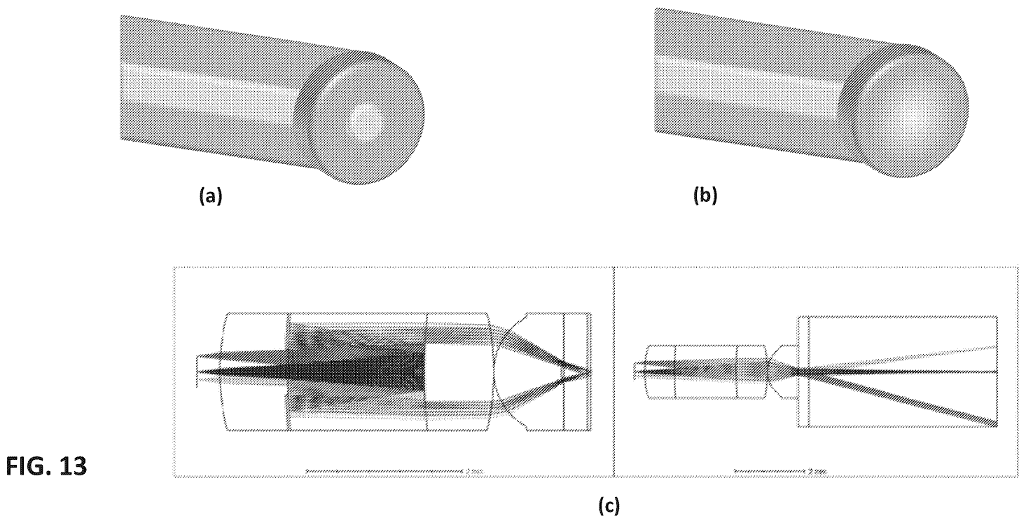

FIG. 13 illustrates three different configurations of a multi-NA lens in accordance with example embodiments. The lens configuration in panel (a) includes a lens configuration with multiple NAs via step changes in lens diameter and/or focal length/radius of curvature. Panel (b) in FIG. 13 illustrates an aspheric configuration with smooth transitions between different NAs via radius of curvature differences corresponding to conic constants. Panel (c) illustrates a triplet lens design that uses dichroic coatings and a telescope design to create two distinct NAs. It should be noted that these designs are provided by the way of example, and not by limitation, to illustrate different techniques and configurations for implementing a multi-NA aperture.

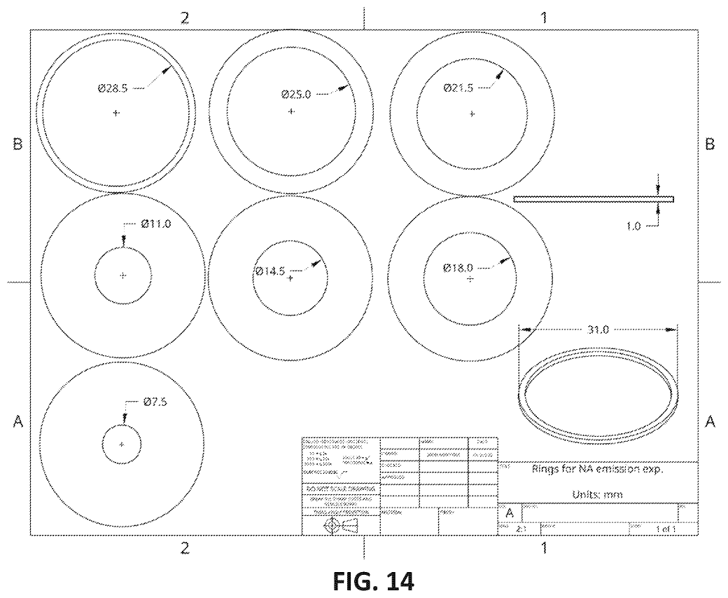

To facilitate experiments using existing systems, collection optics with different NAs can be emulated by modifying the size of an aperture that blocks the light incident on the detectors on the emission side. With reference to FIG. 3, such an aperture can be placed between, for example, the dichroic mirror and the focusing lens that focuses the light onto the detector. Example emission apertures with different aperture sizes are provided in FIG. 14, illustrating different ratios of the central to peripheral sections of the collection optics. In some experiments that use a microscope, changing of the NA can be additionally emulated on the excitation side by changing the distance between two telescope optics that change the diameter of the gaussian (or near gaussian) beam.

Collagen gels can be prepared to emulate colon tissue with different types of colon tissue having different structural characteristics. Collagen fibers are affected by polymerization temperature and pH. For example, the longer you keep the gel at room temperature the thicker the fiber and stronger SHG signal. In a more acidic environment, you get shorter fibers, and in a basic environment, you get longer and thin fibers with weak SHG. The experiments were further designed to work with a laser operating at 780 nm wavelength and constant power at the sample. The measured intensity versus NA for different types of gels can provide the needed information to determine F/B ratio to differentiate between normal and suspicious tissues.

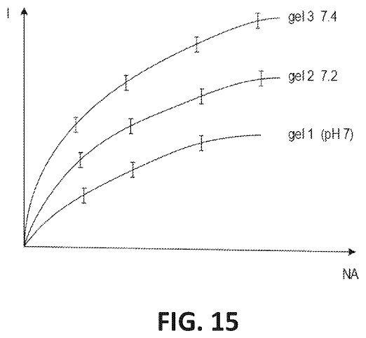

FIG. 15 illustrates one example pattern of the measured intensity versus NA for different collagen gels emulating normal tissue, cancerous tissue, pre-cancerous tissue, etc. By computing and selecting the appropriate F/B ratio (or NA_{high}/NA_{Low}), normal and suspicious tissue can be differentiated without a need to do a paired measurement that requires a “reference” signal for each patient. In vivo measurements can be conducted using an endoscope that includes collection optics with multiple numerical apertures (or collection angles) that allow simultaneous measurement of forward and backward light associated with a region of the colon. The electrical signals associated with the forward and backward light can be processed to determine a measurement like F/B ratio. This ratio can be compared to one or more predetermined values that are indicative of the presence of normal or suspicious tissues. For example, if the F/B ratio is smaller than a particular threshold value, detection of a cancerous region can be indicated. In another example, if the F/B ratio fall between a first and a second threshold (e.g., falls within a range of values), detection of a cancerous region can be indicated. In yet another example, if the F/B ratio is larger than a particular threshold value, the presence of a normal tissue is indicated. In some embodiments, a patient's F/B can be compared to a database of normal F/B ratios to provide a further assessment of the colon tissue.

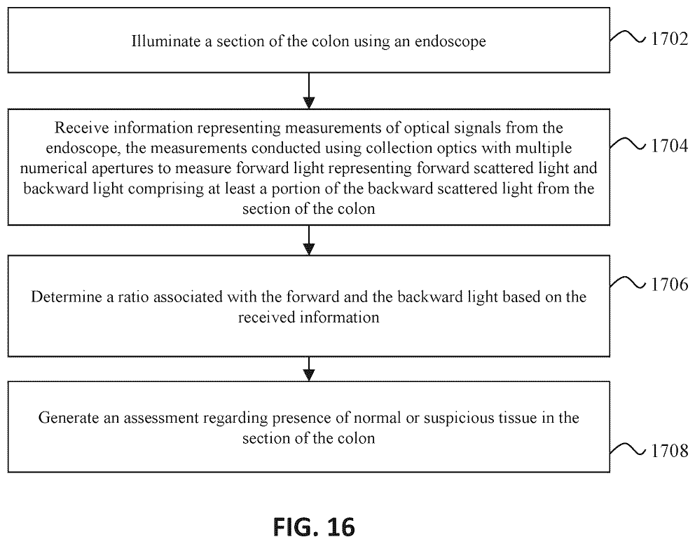

FIG. 16 illustrates a set of operations that can be carried out to determine a presence of a suspicious tissue in a colon in accordance with an example embodiment. At 1602, a section of the colon is illuminated using an endoscope. At 1604, information representing measurements of optical signals from the endoscope is received. The measurements are conducted using collection optics with multiple numerical apertures to measure forward light representing forward scattered light and backward light comprising at least a portion of the backward scattered light from the section of the colon. At 1606, a ratio associated with the forward and the backward light is determined based on the received information. At 1608, an assessment regarding presence of normal or suspicious tissue in the section of the colon is generated.

In one example embodiment, the collection optics has a first and a second numerical aperture configured to collect light associated with the forward light using the first numerical aperture and to collect light associated with the backward light using the second numerical aperture. In another example embodiment, the first numerical aperture is larger than the second numerical aperture. In yet another example embodiment, receiving information representing measurements of optical signals from the endoscope comprises receiving information associated with two or more measurements of the colon, wherein the two or more measurements are obtained in a sequence.

According to another example embodiment, determining the ratio associated with the forward and the backward light includes randomly sampling the received information to obtain a reduced set of information, and using the reduced set of information to determine the ratio. In another example embodiment, generating the assessment regarding the presence of normal or suspicious region includes comparing the ratio associated with the forward and the backward light with one or more threshold values. In yet another example embodiment, upon a determination that the ratio is smaller than a particular threshold value, an indication of the presence of the suspicious region is generated. In still another example embodiment, upon a determination that the ratio falls between a first and a second threshold values, an indication of the presence of a cancerous tissue in the section of the colon is generated. In another example embodiment, upon a determination that the ratio is larger than a particular threshold value, an indication of the presence the normal tissue is generated.

In one aspect of the disclosed embodiment, an endoscopic system is provides that includes an illumination subsection, and a collection subsection configured to collect second harmonic generation (SHG) light from a section of a colon upon illumination by a light source, wherein the collection subsection includes collection optics with at least two different numerical apertures. Another aspect of the disclosed embodiments relates to method for determining a presence of a suspicious tissue that includes using a light source to illuminate the tissue, receiving information representing measurements of optical signals in response to illumination of the tissue, where the measurements are conducted using collection optics with multiple numerical apertures to measure forward light representing forward scattered light and backward light comprising at least a portion of the backward scattered light from the tissue. The methos further includes determining a ratio associated with the forward and the backward light based on the received information. and generating an assessment regarding presence of normal or suspicious regions in the tissue. For example, the tissue is part of a colon, or a tissue with a similar luminal structure.

One aspect of the disclosed embodiments relates to an endoscopic system that includes an excitation subsystem including at least one lens positioned to receive light from a light source and to provide incident light for illuminating a section of a colon. The endoscopic system also includes an emission subsystem including collection optics configured to collect second harmonic generation (SHG) light from the section of the colon; the emission subsystem includes collection optics having multiple numerical apertures (NAs) that allow simultaneous collection of forward light representing forward scattered light and backward light comprising at least a portion of the backward scattered light from the section of the colon. The endoscopic system further includes a processor and a memory including instructions stored thereon. The instructions upon execution by the processor cause the processor to: receive information representing measurements of optical signals associated with the multiple numerical apertures in response to illumination the section of the colon with the incident light, determine a ratio associated with the forward and the backward light based on the received information, and generate an assessment regarding presence of a normal or suspicious region in the section of the colon.

In one example embodiment, the collection optics has a first and a second numerical aperture configured to collect light associated with the forward light using the first numerical aperture and to collect light associated with the backward light using the second numerical aperture. In another example embodiment, the first numerical aperture is larger than the second numerical aperture. In yet another example embodiment, the collection optics includes a multi-NA lens. In still another example embodiment, the multi-NA lens includes a plurality of concentric sections, and at least one of the concentric sections has a higher numerical aperture than another one of the concentric sections. In one example embodiment, the multi-NA lens is an aspheric lens that provides smoothly transitioning numerical apertures from a first numerical aperture to a second numerical aperture.

According to another example embodiment, the excitation subsystem is configured to produce a plurality of excitation numerical apertures to illuminate the section of the colon with different beam widths. In yet another example embodiment, the endoscopic system does not include any internal movable components for scanning the incident light. In still another example embodiment, determination of the ratio associated with the forward and the backward light includes randomly sampling the received information to obtain a reduced set of information, and using the reduced set of information to determine the ratio.

In one example embodiment, generation of the assessment regarding the presence of normal or suspicious region includes comparing the ratio associated with the forward and the backward light with one or more threshold values. In another example, embodiment, the instructions upon execution by the processor cause the processor to, upon a determination that the ratio is smaller than a particular threshold value, generate an indication of the presence of the suspicious region. In still another example embodiment, the instructions upon execution by the processor cause the processor to, upon a determination that the ratio falls between a first and a second threshold values, generate an indication of the presence of a cancerous tissue in the section of the colon. In yet another example embodiment, the instructions upon execution by the processor cause the processor to, upon a determination that the ratio is larger than a particular threshold value, generate an indication of the presence of normal tissue. In one example embodiment, the collection optics includes a multi-NA lens and a multi-clad fiber.

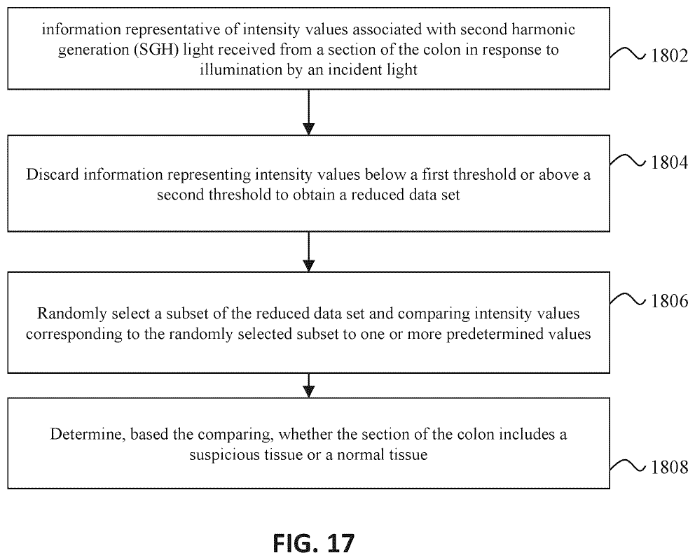

FIG. 17 illustrates a method for identifying a suspicious or a normal tissue in a colon in accordance with an example embodiment. At 1702, information is received that is representative of intensity values associated with second harmonic generation (SHG) light received from a section of the colon in response to illumination by an incident light. At 1704, information representing intensity values below a first threshold or above a second threshold is discarded to obtain a reduced data set. At 1706, a subset of the reduced data set is randomly selected and intensity values corresponding to the randomly selected subset are compared to one or more predetermined values. At 1708, based the comparing, it is determined whether the section of the colon includes a suspicious tissue or a normal tissue.

In one example embodiment, the randomly selected subset consisting of 1000 intensity values is sufficient for determining whether the section of the colon includes a suspicious tissue or a normal tissue. In another example embodiment, discarding the information representing intensity values below the first threshold or above the second threshold consists of one of the following: discarding the information representing intensity values that are below the first threshold, discarding the information representing intensity values that are above the second threshold, or discarding the information representing intensity values that are below the first threshold and discarding information representing intensity values that are above the second threshold.

According to another example embodiment, determining, based the comparing, whether the section of the colon includes the suspicious tissue or the normal tissue is carried out without using an image of the section of the colon. In yet another example embodiment, comparing the intensity values corresponding to the randomly selected subset to one or more predetermined values includes: identifying that the section of the colon includes a suspicious region upon a determination that the intensity values corresponding to the randomly selected subset, on average, exceed a first predetermined value, or identifying that the section of the colon includes a normal region upon a determination that the intensity values corresponding to the randomly selected subset, on average, fall below the first predetermined value but above a second predetermined value.

In still another example embodiment, comparing the intensity values corresponding to the randomly selected subset to one or more predetermined values includes: comparing an average value of the intensity values corresponding to the randomly selected subset to one or more of three predetermined values, and identifying whether the section of the colon includes a normal region, a pre-cancerous region, or a cancerous region based on the comparing. In yet another example embodiment, the above noted method of FIG. 17 further includes, prior to receiving information representative of intensity values associated with the SHG light, illuminating the section of the colon by the incident light using a colonoscope or an endoscope that does not include a moving or scanning mechanism for scanning the incident light over the section of the colon.

Another aspect of the disclosed embodiments relates to an endoscopic system that includes an endoscope configured to be inserted into a colon of a patient, wherein the endoscope is configured to illuminate a section of the colon with incident light and receive second harmonic generation (SHG) light from the section of the colon. The endoscopic system further includes a processor capable of executing computer-readable instructions and a memory comprising computer-readable instructions for: receiving information representative of intensity values associated with the SHG light received from the section of the colon in response to illumination by an incident light, discarding information representing intensity values below a first threshold or above a second threshold to obtain a reduced data set, randomly selecting a subset of the reduced data set and comparing intensity values corresponding to the randomly selected subset to one or more predetermined values; and determining, based on the comparing, whether the section of the colon includes a suspicious tissue or a normal tissue. In one example embodiment, the endoscope does not include a moving or scanning mechanism for scanning the incident light over the section of the colon.

It is understood that the various disclosed embodiments may be implemented individually, or collectively, using devices comprised of various optical components, electronics hardware and/or software modules and components. These devices, for example, may comprise a processor, a memory unit, an interface that are communicatively connected to each other, and may range from desktop and/or laptop computers, to mobile devices and the like. The processor and/or controller can perform various disclosed operations based on execution of program code that is stored on a storage medium. The processor and/or controller can, for example, be in communication with at least one memory and with at least one communication unit that enables the exchange of data and information, directly or indirectly, through the communication link with other entities, devices and networks. The communication unit may provide wired and/or wireless communication capabilities in accordance with one or more communication protocols, and therefore it may comprise the proper transmitter/receiver antennas, circuitry and ports, as well as the encoding/decoding capabilities that may be necessary for proper transmission and/or reception of data and other information. For example, the processor may be configured to determine the F/B ratio or other computations based on the techniques disclosed herein.

Various information and data processing operations described herein may be implemented in one embodiment by a computer program product, embodied in a computer-readable medium, including computer-executable instructions, such as program code, executed by computers in networked environments. A computer-readable medium may include removable and non-removable storage devices including, but not limited to, Read Only Memory (ROM), Random Access Memory (RAM), compact discs (CDs), digital versatile discs (DVD), etc. Therefore, the computer-readable media that is described in the present application comprises non-transitory storage media. Generally, program modules may include routines, programs, objects, components, data structures, etc. that perform particular tasks or implement particular abstract data types. Computer-executable instructions, associated data structures, and program modules represent examples of program code for executing steps of the methods disclosed herein. The particular sequence of such executable instructions or associated data structures represents examples of corresponding acts for implementing the functions described in such steps or processes.

The foregoing description of embodiments has been presented for purposes of illustration and description. The foregoing description is not intended to be exhaustive or to limit embodiments of the present invention to the precise form disclosed, and modifications and variations are possible in light of the above teachings or may be acquired from practice of various embodiments. The embodiments discussed herein were chosen and described in order to explain the principles and the nature of various embodiments and its practical application to enable one skilled in the art to utilize the present invention in various embodiments and with various modifications as are suited to the particular use contemplated. While operations are depicted in the drawings in a particular order, this should not be understood as requiring that such operations be performed in the particular order shown or in sequential order, or that all illustrated operations be performed, to achieve desirable results. The features of the embodiments described herein may be combined in all possible combinations of methods, apparatus, modules, and systems.

1. An endoscopic system, comprising:
an excitation subsystem including at least one lens positioned to receive light from a light source and to provide incident light for illuminating a section of a colon;
an emission subsystem including collection optics configured to collect second harmonic generation (SHG) light from the section of the colon, the emission subsystem including collection optics having multiple numerical apertures (NAs) that allow simultaneous collection of forward light representing forward scattered light and backward light comprising at least a portion of the backward scattered light from the section of the colon;
a processor and a memory including instructions stored thereon, wherein the instructions upon execution by the processor cause the processor to:
receive information representing measurements of optical signals associated with the multiple numerical apertures in response to illumination the section of the colon with the incident light,
determine a ratio associated with the forward and the backward light based on the received information, and
generate an assessment regarding presence of a normal or suspicious region in the section of the colon.

2. The endoscopic system of claim 1, wherein:
the collection optics has a first and a second numerical aperture configured to collect light associated with the forward light using the first numerical aperture and to collect light associated with the backward light using the second numerical aperture.

3. The endoscopic system of claim 2, wherein the first numerical aperture is larger than the second numerical aperture.

4. The endoscopic system of claim 1, wherein the collection optics includes a multi-NA lens.

5. The endoscopic system of claim 4, wherein:
the multi-NA lens includes a plurality of concentric sections, and
at least one of the concentric sections has a higher numerical aperture than another one of the concentric sections.

6. The endoscopic system of claim 4, wherein:
the multi-NA lens is an aspheric lens that provides smoothly transitioning numerical apertures from a first numerical aperture to a second numerical aperture.

7. The endoscopic system of claim 1, wherein the excitation subsystem is configured to produce a plurality of excitation numerical apertures to illuminate the section of the colon with different beam widths.

8. The endoscopic system of claim 1, wherein the endoscopic system does not include any internal movable components for scanning the incident light.

9. The endoscopic system of claim 1, wherein determination of the ratio associated with the forward and the backward light includes randomly sampling the received information to obtain a reduced set of information, and using the reduced set of information to determine the ratio.

10. The endoscopic system of claim 1, wherein generation of the assessment regarding the presence of normal or suspicious region includes comparing the ratio associated with the forward and the backward light with one or more threshold values.

11. The endoscopic system of claim 10, wherein the instructions upon execution by the processor cause the processor to, upon a determination that the ratio is smaller than a particular threshold value, generate an indication of the presence of the suspicious region.

12. The endoscopic system of claim 10, wherein the instructions upon execution by the processor cause the processor to, upon a determination that the ratio falls between a first and a second threshold values, generate an indication of the presence of a cancerous tissue in the section of the colon.

13. The endoscopic system of claim 10, wherein the instructions upon execution by the processor cause the processor to, upon a determination that the ratio is larger than a particular threshold value, generate an indication of the presence of normal tissue.

14. The endoscopic system of claim 1, wherein the collection optics includes a multi-NA lens and a multi-clad fiber.

15. A method for determining a presence of a suspicious tissue in a colon, comprising:
using an endoscope to illuminate a section of the colon;
receiving information representing measurements of optical signals from the endoscope, the measurements conducted using collection optics with multiple numerical apertures to measure forward light representing forward scattered light and backward light comprising at least a portion of the backward scattered light from the section of the colon;
determining a ratio associated with the forward and the backward light based on the received information; and
generating an assessment regarding presence of normal or suspicious tissue in the section of the colon.

16. The method of claim 15, wherein
the collection optics has a first and a second numerical aperture configured to collect light associated with the forward light using the first numerical aperture and to collect light associated with the backward light using the second numerical aperture.

17. The method of claim 16, wherein the first numerical aperture is larger than the second numerical aperture.

18. The method of claim 15, wherein receiving information representing measurements of optical signals from the endoscope comprises receiving information associated with two or more measurements of the colon, wherein the two or more measurements are obtained in a sequence.

19. The method of claim 15, wherein determining the ratio associated with the forward and the backward light includes randomly sampling the received information to obtain a reduced set of information, and using the reduced set of information to determine the ratio.

20. The method of claim 15, wherein generating the assessment regarding the presence of normal or suspicious region includes comparing the ratio associated with the forward and the backward light with one or more threshold values.

21. The method of claim 20, wherein, upon a determination that the ratio is smaller than a particular threshold value, generating an indication of the presence of the suspicious region.

22. The method of claim 20, wherein, upon a determination that the ratio falls between a first and a second threshold values, generating an indication of the presence of a cancerous tissue in the section of the colon.

23. The method of claim 20, wherein, upon a determination that the ratio is larger than a particular threshold value, generating an indication of the presence the normal tissue.

24. A method for determining a presence of a suspicious tissue, comprising:
using a light source to illuminate the tissue;
receiving information representing measurements of optical signals in response to illumination of the tissue, the measurements conducted using collection optics with multiple numerical apertures to measure forward light representing forward scattered light and backward light comprising at least a portion of the backward scattered light from the tissue;
determining a ratio associated with the forward and the backward light based on the received information; and
generating an assessment regarding presence of normal or suspicious regions in the tissue.

25. The method of claim 24, wherein the tissue is part of a colon.

26. An endoscope, comprising:
an illumination subsection, and
a collection subsection configured to collect second harmonic generation (SHG) light from a section of a colon upon illumination by a light source, the collection subsection including collection optics with at least two different numerical apertures.

27. A method for identifying a suspicious or a normal tissue in a colon, comprising:
receiving information representative of intensity values associated with second harmonic generation (SHG) light received from a section of the colon in response to illumination by an incident light;
discarding information representing intensity values below a first threshold or above a second threshold to obtain a reduced data set;
randomly selecting a subset of the reduced data set and comparing intensity values corresponding to the randomly selected subset to one or more predetermined values; and
determining, based the comparing, whether the section of the colon includes a suspicious tissue or a normal tissue.

28. The method of claim 27, wherein the randomly selected subset consisting of 1000 intensity value is sufficient for determining whether the section of the colon includes a suspicious tissue or a normal tissue.

29. The method of claim 27, wherein discarding the information representing intensity values below the first threshold or above the second threshold consists of one of the following:
discarding the information representing intensity values that are below the first threshold,
discarding the information representing intensity values that are above the second threshold, or
discarding the information representing intensity values that are below the first threshold and discarding information representing intensity values that are above the second threshold.

30. The method of claim 27, wherein determining, based the comparing, whether the section of the colon includes the suspicious tissue or the normal tissue is carried out without using an image of the section of the colon.

31. The method of claim 27, wherein comparing the intensity values corresponding to the randomly selected subset to one or more predetermined values includes:
identifying that the section of the colon includes a suspicious region upon a determination that the intensity values corresponding to the randomly selected subset, on average, exceed a first predetermined value, or
identifying that the section of the colon includes a normal region upon a determination that the intensity values corresponding to the randomly selected subset, on average, fall below the first predetermined value but above a second predetermined value.

32. The method of claim 27, wherein comparing the intensity values corresponding to the randomly selected subset to one or more predetermined values includes:
comparing an average value of the intensity values corresponding to the randomly selected subset to one or more of three predetermined values, and
identifying whether the section of the colon includes a normal region, a pre-cancerous region, or a cancerous region based on the comparing.

33. The method of claim 27, further comprising, prior to receiving information representative of intensity values associated with the SHG light, illuminating the section of the colon by the incident light using a colonoscope or an endoscope that does not include a moving or scanning mechanism for scanning the incident light over the section of the colon.

34. An endoscopic system, comparing:
an endoscope configured to be inserted into a colon of a patient, wherein the endoscope is configured to illuminate a section of the colon with incident light and receive second harmonic generation (SHG) light from the section of the colon;
a processor capable of executing computer-readable instructions and a memory comprising computer-readable instructions for:
receiving information representative of intensity values associated with the SHG light received from the section of the colon in response to illumination by an incident light;
discarding information representing intensity values below a first threshold or above a second threshold to obtain a reduced data set;
randomly selecting a subset of the reduced data set and comparing intensity values corresponding to the randomly selected subset to one or more predetermined values; and
determining, based the comparing, whether the section of the colon includes a suspicious tissue or a normal tissue.

35. The endoscopic system of claim 34, wherein the endoscope does not include a moving or scanning mechanism for scanning the incident light over the section of the colon.
